(19) United States
(12) Patent Application Publication
(10) Pub. No.: US 2023/0414612 A1
(43) Pub. Date: Dec. 28, 2023

(54) PHARMACEUTICAL COMPOSITION COMPRISING A CAP-DEPENDENT ENDONUCLEASE INHIBITOR

(71) Applicants: TaiGen Biotechnology Co., Ltd., Taipei (TW); TaiGen Biopharmaceuticals Co.(Beijing), Ltd., Beijing (CN)

(72) Inventors: Chung-Shu Hong, Taipei (TW); Wei-Tsung Chen, Taipei (TW)

(21) Appl. No.: 18/341,946

(22) Filed: Jun. 27, 2023

Related U.S. Application Data
(60) Provisional application No. 63/355,742, filed on Jun. 27, 2022.

(51) Int. Cl. A61K 31/502; A61K 9/14; A61K 47/38

(52) U.S. Cl. CPC A61K 31/502; A61K 9/146; A61K 47/38

(57) ABSTRACT

The present disclosure provides a pharmaceutical composition including a solid dispersion containing a cap-dependent endonuclease inhibitor or a pharmaceutically acceptable salt thereof for oral administration.

### REFERENCE TO RELATED APPLICATION

This application claims the benefit of the filing date under 35 U.S.C. 119(e) to U.S. Provisional Patent Application No. 63/355,742, filed on Jun. 27, 2022, the entire contents of which are incorporated herein by reference.

### BACKGROUND

### 1. Technical Field

The present disclosure relates to a pharmaceutical composition containing a heterocyclic compound or a pharmaceutically acceptable salt thereof.

### 2. Description of Related Art

Cap-dependent endonuclease is an essential enzyme in the inhibition of mRNA synthesis of influenza viruses. Inhibitors of Cap-dependent endonuclease are found to be effectively against influenza virus A and B. Several compounds displayed potent antiviral activity against influenza virus by inhibiting Cap-dependent endonuclease. In PCT published application, WO2019/144089, novel heterocyclic compounds as potent Cap-dependent endonuclease inhibitors were first disclosed. Effective therapeutic interventions often rely on the ability to deliver pharmaceutical compounds in a convenient and patient-friendly manner. Oral administration is the preferred route for drug delivery due to its non-invasiveness, ease of administration, and patient compliance. The aforementioned novel anti-influenza heterocyclic compounds shown substantial promise in preclinical studies. However, its oral administration presents several challenges including low solubility and poor stability. Therefore, developing optimal pharmaceutical formulations is crucial to ensure the compound's therapeutic efficacy. The formulation of the present disclosure aims to improve solubility, enhance patient compliance, and maximize therapeutic benefits across different age groups.

### SUMMARY

The present disclosure is to provide a pharmaceutical composition comprising a solid dispersion. The solid dispersion includes a compound of Formula (II) or a pharmaceutically acceptable salt thereof, and a pharmaceutically acceptable polymer; a weight ratio of the compound of Formula (II) or a pharmaceutically acceptable salt thereof to the pharmaceutically acceptable polymer is from about 1:1 to about 1:5; and the compound of Formula (II) or a pharmaceutically acceptable salt thereof is in a therapeutically effective amount from about 5 mg to about 200 mg. In the compound of Formula (II), G is hydrogen or —C(R₂R₂′)—O—CO—O—R₃, in which each of R₂, and R₂′, independently, is hydrogen or C₁₋₄ alkyl; R₃ is C₁₋₄ alkyl; the star (*) indicates a chiral center.

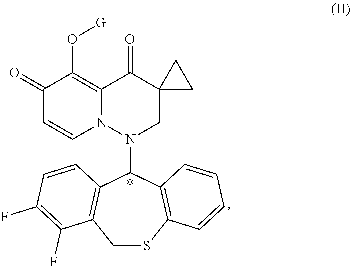

Also provided herein is a process for preparing said pharmaceutical composition comprises a solid dispersion of the compound of Formula (II) or a pharmaceutically acceptable salt thereof.

Further provided herein is a method for treating influenza, comprising administering a pharmaceutical composition comprising a therapeutically effective amount of the compound of Formula (II) or a pharmaceutically acceptable salt thereof to a subject in need thereof. Also provided herein is the pharmaceutical composition for use in treating influenza. In addition, the preset disclosure provides a use of the pharmaceutical composition in the manufacture of a medicament for treating influenza.

### BRIEF DESCRIPTION OF THE DRAWING

The present disclosure can be more fully understood by reading the following description of the embodiments, with reference made to the accompanying drawings.

FIG. 1 is the dissolution profile of granule formulations and ASD powder itself.

FIG. 2 is the dissolution profile of oral disintegrating formulation and ASD powder itself.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

The term “about” means within an acceptable error range for the particular value as determined by persons of ordinary skill in the art, which will depend in part on how the value is measured or determined, i.e., the limitations of the measurement system. As used herein, when referring to a measurable or calculated value such as an amount, a temporal duration, a concentration, a ratio, and the like, may encompass variations of ±20%, ±10%, ±5%, ±1%, or ±0.1% from the specified value, as such variations are appropriate to perform the disclosed methods.

The term “treat” is meant to include alleviating or abrogating a disorder, disease, or condition, or one or more of the symptoms associated with the disorder, disease, or condition; or alleviating or eradicating the cause(s) of the disorder, disease, or condition itself.

The term “prevent” is meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “patient”, “individual” or “subject” refer to a human or a non-human mammal. In one embodiment, the patient, individual, or subject is human. In another embodiment, the patient, individual, or subject is children.

The term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

The term “one or more” refers to either one or a number above one (e.g., 2, 3, 4, 5, 6, 7 or above).

The term “C₁₋₄ alkyl” refers to a straight- or branched-chain saturated hydrocarbyl substituent containing 1 to 4 carbon atoms. Examples of C₁₋₄ alkyl include methyl, ethyl, n-propyl, isopropyl, n-butyl, isobutyl, sec-butyl, tert-butyl and the like, but are not limited thereto.

The term “C₁₋₄ alkoxy” refers to the group —OR wherein R is C₁₋₄ alkyl. Examples of C₁₋₄ alkoxy include methoxy, ethoxy, n-propoxy, isopropoxy, n-butoxy, iso-butoxy, sec-butoxy, and tert-butoxy, but are not limited thereto.

The term “C₁₋₄ alkylamine” refers to the group —NHR′ wherein R′ is C₁₋₄ alkyl. Examples of C₁₋₄ alkylamine include methylamino, ethylamino, and isopropylamino, but are not limited thereto.

The term “carbocyclyl” refers to C₃ to C₁₀ cyclic hydrocarbon group including an aromatic carbocyclyl and/or a non-aromatic carbocyclyl.

The term “aromatic carbocyclyl” refers to a cyclic aromatic hydrocarbon group which is monocyclic or polycyclic having two or more rings. Examples of aromatic carbocyclyl include phyeny, naphthyl, anthryl, phenanthryl and the like, but are not limited thereto.

The term “non-aromatic carbocyclyl” refers to a cyclic hydrocarbon that is completely saturated or that contains one or more units of unsaturation but which is not aromatic.

The term “heterocyclyl” includes an aromatic heterocyclyl and a non-aromatic heterocyclyl, which contains one or more of heteroatom(s) independently selected from the group consisting of O, S, N, and any combination thereof.

The term “aromatic heterocyclyl” refers to an aromatic cyclyl, which is a monocyclic or a polycyclic having two or more rings, containing one or more of heteroatom(s) independently selected from the group consisting of O, S, N, and any combination thereof.

The term “non-aromatic heterocyclyl” refers to a non-aromatic cyclyl, which is a monocyclic or a polycyclic having two or more rings containing one or more heteroatom(s) independently selected from the group consisting of O, S, N, and any combination thereof.

The term “pharmaceutically acceptable salt” encompasses non-toxic acid and base addition salts or the compound to which the term refers. The pharmaceutically acceptable salts include those derived from organic and inorganic acids or bases know in the art.

The term “solid dispersion” refers to a molecular dispersion of a compound, particularly a drug substance within a pharmaceutically acceptable carrier (such as polymer). The term “solid dispersion” in general means a system in a solid state comprising at least two components, wherein one component is dispersed substantially evenly throughout the other component(s). For example, solid dispersions may be the dispersion of one or more active ingredients in an inert carrier or matrix at solid state, prepared by the spray drying, hot melt extrusion, fluid bed, or lyophilization methods. Formation of a molecular dispersion may provide a means of reducing the particle size to nearly molecular levels.

As used herein, a polyvinyl pyrrolidone (also called PVP) refers to a polymer compound obtained by polymerizing N-vinyl-2-pyrrolidone. The polyvinyl pyrrolidone includes for example but is not limited to PVP-K17, PVP-K25, PVP-K30, PVP-K40, PVP-K50, PVP-K60, PVP-K70, PVP-K80, PVP-K85, PVP-K90, PVP-K120, and the like, but are not limited thereto.

As used herein, a polyvinylpyrrolidone-vinylacetate (also called PVP-VA) refers to the copolymer of vinylpyrrolidone (VP) and vinylacetate (VA) monomers. Examples of the polyvinylpyrrolidone-vinylacetate copolymer include PVP-VA64, Kollidon SR, and the like, but are not limited thereto.

As used herein, a methacrylic acid methyl methacrylate copolymer refers to a copolymer derived from esters of acrylic and methacrylic acid. Eudragit is the brand name for a diverse range of the methacrylic acid methyl methacrylate copolymers. Examples of the methacrylic acid methyl methacrylate copolymer include Eudragit EPO, Eudragit E100, Eudragit RS100, Eudragit RL100, Eudragit L100, Eudragit NE, Eudragit NM, Eudragit FS, and the like, but are not limited thereto.

As used herein, a polyethylene glycol (also called PEG) refers to a polymer containing ethylene glycol monomer units of formula —O—CH₂—CH₂—. Examples of the polyethylene glycol include PEG-1000, PEG-1500, PEG-2000, PEG-2500, PEG-3000, PEG-3350, PEG-3500, PEG-4000, PEG-5000, PEG-6000, PEG-8000, and the like, but are not limited thereto.

As used herein, a polyoxyethylene-polyoxypropylene copolymer refers to a block copolymer, one block is polyoxyethylene, and the other block is polyoxypropylene. Examples of the polyoxyethylene-polyoxypropylene copolymer include the Pluronic® series of surfactants (available from BASF). For example but not limited thereto, the Pluronic® series of surfactants can be designated by the CTFA name of Poloxamer 108, 124, 188, 217, 237, 238, 288, 338, 407, 101, 105, 122, 123, 181, 182, 183, 184, 212, 231, 282, 331, 401, 402, 185, 215, 234, 235, 284, 333, 334, 335, and 403.

As used herein, a hydroxypropyl cellulose is also called HPC. Examples of the hydroxypropyl cellulose include HPC-SSL, HPC-SL, HPC-L, HPC-M, and HPC-H according to the average molecular weight.

As used herein, a hydroxypropyl methyl cellulose is also called HPMC. Examples of the hydroxypropyl methyl cellulose include E3, E5, E6, E15, E50Lv, etc., depending on the viscosity, but are not limited thereto.

As used herein, a hydroxypropyl methyl cellulose acetate succinate (also called HPMCAS) refers to a mixture of acetic acid and monosuccinic acid esters of hydroxypropylmethyl cellulose. In one embodiment, the HPMCAS comprises various types, such as LF, LG, MF, MG, HF and HG, but are not limited thereto.

As used herein, a hydroxypropyl methyl cellulose phthalate also called HPMCP. Examples of HPMCP include HPMCP HP-50, HPMCP HP-55 and HPMCP HP-55S, but are not limited thereto.

The term “amorphous” refers to a solid form of a molecule that is not crystalline. In particular, an amorphous solid dose not display a definite X-ray diffraction pattern.

The term “therapeutically effective amount” refers to the amount or dose of an active compound or salt thereof that is sufficient to prevent development of, or alleviate to some extent, one or more of the symptoms of the disorder, disease, or condition being treated. Compound A (5 mg/kg, oral) of this disclosure was tested in vivo influenza virus (A/PR/8/34) infection mouse (BALB/c) model and showed good antiviral efficacy on survival rate and body weight change. In addition, the effect of Compound A on influenza virus growth in lungs of BALB/c mice infected with low infectious dose 100 pfu/mouse of A/PR/8/34 at one-day post-infection was conducted. Virus titers in Compound A treated groups (5 and 25 mg/kg) were significantly lower than those in vehicle treated group. When the dosage levels were set at 300 mg/kg/day of Compound A in 14-day repeated-dose oral toxicity study, Compound A did not cause deaths or have toxic effect. Therefore, based on the results of these preliminary in vivo animal studies, it is estimated that the reasonable therapeutically effective amount of the compound of Formula (I) or a pharmaceutically acceptable salt thereof for human can be about 5-200 mg, about 5-150 mg, about 5-100 mg, about 10-80 mg, about 10-40 mg, about 10-30 mg, about 10-20 mg, about 5-10 mg, about 5-20 mg, about 20-40 mg, or about 40-80 mg. For example but not limited thereto, the therapeutically effective amount of the compound of Formula (I) or a pharmaceutically acceptable salt thereof is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg.

The disclosure provided herein relates to a pharmaceutical composition comprising a solid dispersion, wherein the solid dispersion comprises:
i) a compound of Formula (I) or a pharmaceutically acceptable salt thereof:

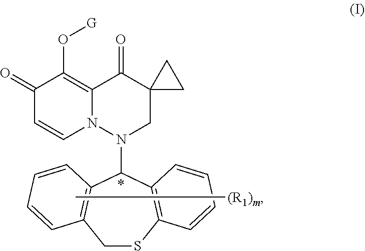

wherein G is hydrogen, a prodrug group or a suitable substituted group; R₁ is halogen, C₁₋₄ alkyl, or deuterium; m is an integer of 1 to 9; the star (*) indicates a chiral center; and
ii) a pharmaceutically acceptable polymer,
wherein a weight ratio of the compound of Formula (I) or a pharmaceutically acceptable salt thereof to the pharmaceutically acceptable polymer is from about 1:1 to about 1:5, and the compound of Formula (I) or a pharmaceutically acceptable salt thereof is in a therapeutically effective amount from about 5 mg to about 200 mg, about 5 mg to about 150 mg, about 5 mg to about 100 mg, about 10 mg to about 80 mg, about 10 mg to about 40 mg, about 10 mg to about 30 mg, about 10 mg to about 20 mg, about 5 mg to about 10 mg, about 5 mg to about 20 mg, about 20 mg to about 40 mg, or about 40 mg to about 80 mg.

In one embodiment, R₁ is halogen, and m is an integer of 1 to 3. In another embodiment, R₁ is fluoro, and m is an integer of 1 or 2.

A method for treating influenza in a patient in a need thereof, comprises administering an oral pharmaceutical composition of the present disclosure. In one embodiment, the method comprises administering to the patient about 1 mg/kg of the compound of Formula (I) or a pharmaceutically acceptable salt thereof in a dosage form. In another embodiment, the method comprises administering to the patient about 2 mg/kg of the compound of Formula (I) or a pharmaceutically acceptable salt thereof in a dosage form.

In one embodiment, the cyclic group in Formula (I) is represented by the formula:

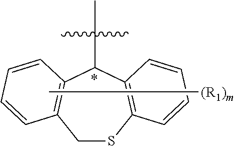

is a cyclic group represented by any one of the following structures:

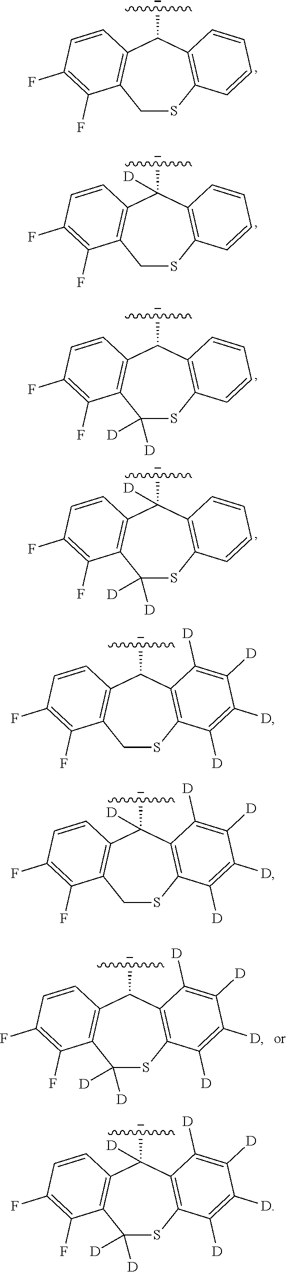

In one embodiment, the compound of Formula (I) is represented by Formula (II):

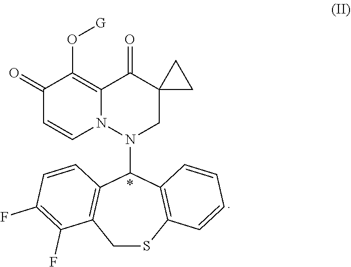

In one embodiment, G is selected from the group consisting of hydrogen, —C(R₂R₂′)—O—CO—R₃, —C(R₂R₂′)—O—CO—O—R₃, —C(R₂R₂′)—NR₄—C(═O)—CO—O—R₃, —C(R₂R₂′)—O—CO—C(R₂R₂′)—NR₄—CO—O—R₃, —C(R₂R₂′)—C(R₂R₂′)—O—CO—R₃, —C(R₂R₂′)—R₃, —C(═O)—O—R₃, —C(═O)—R₃, —C(═O)—O-alkylene-O—R₃, —C(═O)—NR₃R₄, —(CH₂)₂—OH, —(CH₂)₃—OH, —(CH₂)₂—O—SO₂R₅, —(CH₂)₂—O—P(═O)(R₅R₆) and —P(═O)(R₅R₆), in which each of R₂, R₂′, and R₄, independently, is hydrogen or C₁₋₈ alkyl; R₃ is C₁₋₄ alkyl, C₃₋₁₀ carbocyclyl, or C₃₋₁₀ heterocyclyl; R₅ is OH, NH2, C₁₋₄ alkyl or C₁₋₄ alkoxy; and R₆ is OH, C₁₋₄ alkoxy or C₁₋₄ alkylamine. In another embodiment, G is hydrogen or —C(R₂R₂′)—O—CO—O—R₃. In one embodiment, each of R₂, and R₂′, independently, is hydrogen or C₁₋₈ alkyl. In another embodiment, each of R₂, and R₂′, independently, is hydrogen or C₁₋₄ alkyl.

In one embodiment, the G group is selected from the group consisting of hydrogen,

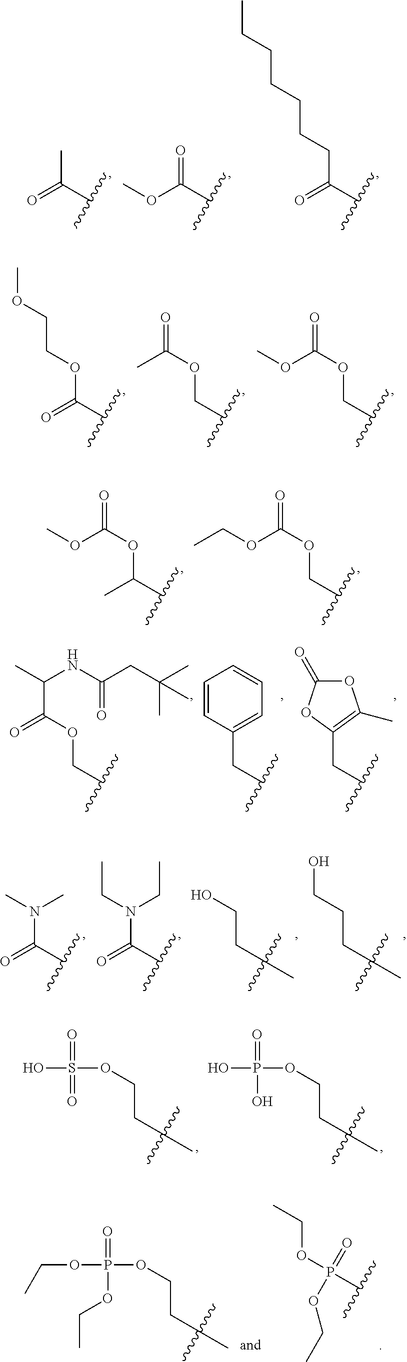

In one embodiment, the compound of Formula (I) is [1-((11S)-7,8-difluoro(6H,11H-dibenzo[c,f]thiepin-11-yl))-4,6-dioxospiro[1,2,3,9-tetrahydropyridino[1,2-e]pyridazine-3,1′-cyclopropane]-5-yloxy]methyl methoxyformate, or its metabolite, 1′-((11S)-7,8-difluoro-6,11-dihydrodibenzo[b,e]thiepin-11-yl)-1′,2′-dihydro-5′-hydroxy-spiro[cyclopropane-1,3′-(3H)pyrido[1,2-b]pyridazine-4′,6′-dione, or a pharmaceutically acceptable salt thereof.

In some embodiments, the compounds of the present disclosure can be prepared according to the methods/procedures disclosed in PCT published application No. WO2019/144089, or No. WO2021/239126, which is incorporated herein by reference in its entirety.

In one embodiment, the disclosure provides an amorphous solid dispersion of a compound of Formula (I) or a pharmaceutically acceptable salt thereof, further comprising a pharmaceutically acceptable polymer.

In another embodiment, disclosed herein is an amorphous solid dispersion comprising a compound of Formula (I) or a pharmaceutically acceptable salt thereof, and a pharmaceutically acceptable polymer, wherein a compound of Formula (I) or a pharmaceutically acceptable salt thereof is molecularly dispersed within a polymer matrix formed by the pharmaceutically acceptable polymer in its solid state.

The pharmaceutically acceptable polymer uses in the amorphous solid dispersion of the present disclosure is a water-soluble polymer. The suitable water-soluble polymer should act as a water-soluble carrier to make the active ingredient hydrophilic, thereby improving its solubility, and it also helps maintaining the solid dispersion in an amorphous state. Some common examples of the water-soluble polymer include but not limited to vinyl polymers and copolymers, polyvinyl pyrrolidone (PVP), polyvinylpyrrolidone-vinylacetate copolymer (PVP-VA), polyvinyl alcohols (PVA), polyvinyl alcohol polyvinyl acetate copolymers, polyethylene polyvinyl alcohol copolymers, polyvinyl caprolactam and polyvinyl acetate, polyvinyl acetate phthalate (PVAP), polyvinyl caprolactam-polyvinyl acetate-polyethylene glycol graft copolymer (such as Soluplus), acrylate and methacrylate copolymers, methacrylic acid methyl methacrylate copolymer (such as Eudragit), polyethylene glycol (PEG), polyoxyethylene-polyoxypropylene copolymers (also referred to as poloxamers), hydroxypropyl methyl cellulose acetate (HPMCA), hydroxypropyl methyl cellulose (HPMC), hydroxypropyl cellulose (HPC), methyl cellulose, hydroxyethyl methyl cellulose, hydroxyethyl cellulose, hydroxyethyl cellulose acetate, hydroxyethyl ethyl cellulose, hydroxypropyl methyl cellulose acetate succinate (HPMCAS), hydroxypropyl methyl cellulose phthalate (HPMCP), carboxymethylethyl cellulose (CMEC), cellulose acetate phthalate (CAP), cellulose acetate succinate (CAS), hydroxypropyl methyl cellulose acetate phthalate (HPMCAP), cellulose acetate trimellitate (CAT), hydroxypropyl methyl cellulose acetate trimellitate (HPMCAT), carboxymethyl cellulose acetate butyrate (CMCAB), calcium carboxymethyl cellulose (calcium CMC), sodium carboxymethyl cellulose (sodium CMC), β-cyclodextrin, hydroxypropyl-β-cyclodextrin, sulfobutylether-β-cyclodextrin, docusate sodium, cellacefate or the like.

In one embodiment, the pharmaceutically acceptable polymer is polyvinylpyrrolidone-vinylacetate copolymer (PVP-VA), polyvinyl caprolactam-polyvinyl acetate-polyethylene glycol graft copolymer (Soluplus), methylacrylic acid methyl methacrylate copolymer (such as Eudragit), hydroxypropyl cellulose (HPC), hydroxypropyl methyl cellulose acetate succinate (HPMCAS), hydroxypropyl methylcellulose phthalate (HPMCP), or mixtures thereof.

In one embodiment, the pharmaceutically acceptable polymer is polyvinyl caprolactam-polyvinyl acetate-polyethylene glycol graft copolymer, hydroxypropyl cellulose (HPC), hydroxypropyl methyl cellulose acetate succinate (HPMCAS), or mixtures thereof. In another embodiment, the pharmaceutically acceptable polymer is HPC, wherein the HPC comprises HPC-SSL, HPC-SL, HPC-L, HPC-M, or HPC-H.

In one embodiment, the pharmaceutically acceptable polymer is Soluplus, HPC-SSL, HPMCAS, or mixtures thereof.

In one embodiment, disclosed herein is an amorphous solid dispersion comprising a compound of Formula (I) or a pharmaceutically acceptable salt thereof and a pharmaceutically acceptable polymer, wherein the weight ratio of a compound of Formula (I) or a pharmaceutically acceptable salt thereof to the pharmaceutically acceptable polymer is in the range of 4:1 to 1:7, 2:1 to 1:7, 3:1 to 1:6, 2.5:1 to 1:6, 2:1 to 1:5, 2:1 to 1:4, 2:1 to 1:3.5, 2:1 to 1:2, 2:1 to 1:1.5, 2:1 to 1:5, 1.5:1 to 1:5, 1.5:1 to 1:3.5, 1.5:1 to 1:2, 1.5:1 to 1:1.5, 1:1 to 1:5, 1:1 to 1:3.5, 1:1 to 1:3, 1:1 to 1:2.5, 1:1 to 1:2, 1:1 to 1:1.5. In one embodiment, the weight ratio is about 1:1 to about 1:5. In another embodiment, the weight ratio is about 1:1 to about 1:3.5. In still another embodiment, the weight ratio is about 1:1 to about 1:3. In other embodiment, the weight ratio is about 1:3.

In one embodiment, the amount of the compound of Formula (I) or a pharmaceutically acceptable salt thereof in the solid dispersion is in general 10-60%, 10-55%, 10-50%, 10-45%, 10-40%, 15-60%, 15-55%, 15-50%, 15-45%, or 15-40% by weight. For example, the amount of the compound of Formula (I) or a pharmaceutically acceptable salt thereof in solid dispersion (drug loading) is about 15%, 25%, 33%, 40% or 50% by weight.

In one embodiment, the solid dispersion of the present disclosure has a D₅₀ particle size in the range of about 4 μm to about 15 μm, or a D₉₀ particle size in the range of about 15 μm to about 50 μm.

The solid dispersion described herein can be administered orally to a subject (e.g., a human) in need thereof to treat or prevent the infectious diseases such as influenza.

The amorphous solid dispersion of this disclosure may be prepared by methods well known in the art, such as spray drying, hot melt extrusion, fluid bed, or lyophilization technique. In one embodiment, the amorphous solid dispersion is prepared by spray drying.

In one embodiment, the amorphous solid dispersion of this disclosure is prepared by dissolving a compound of Formula (I) or a pharmaceutically acceptable salt thereof in a sufficient amount of an organic solvent, and mixing the resultant solution with a solution containing a pharmaceutically acceptable polymer, thereby preparing a spray solution. The solvent may then be evaporated away, leaving the drug dispersed/dissolved in the matrix. Any organic solvent that can dissolve or disperse a compound of Formula (I) or a pharmaceutically acceptable salt thereof and the pharmaceutical acceptable polymer described above can be used. Examples of the organic solvent include lower carbon-number alcohols (such as methanol, ethanol, propanol, or isopropanol), ketone (such as acetone, methyl ethyl ketone or methyl isobutyl ketone), haloalkane (such as dichloromethane, chloroform or carbon tetrachloride), acetic acid, ethyl acetate, N,N-dimethylformamide, DMSO, tetrahydrofuran, or mixtures thereof.

In one embodiment, the preparation of the amorphous solid dispersion comprises the steps of: (i) dissolving a compound of Formula (I) or a pharmaceutically acceptable salt thereof, and a pharmaceutically acceptable polymer in a solvent, and (ii) drying the solution obtained in step (i).

In one embodiment, step (i) comprises: dissolving a compound of Formula (I) or a pharmaceutically acceptable salt thereof in a sufficient amount of an organic solvent; dissolving a pharmaceutically acceptable polymer in a solvent; and mixing the two solutions.

In one embodiment, step (ii) comprises spray drying. In another embodiment, step (ii) comprises spray drying in combination with a fluid bed. In a further embodiment, step (ii) comprises evaporation of the solvent using a rotovap.

In one embodiment, the solvent may be removed by evaporation by spray drying technique. The term “spray drying” is used conventionally and broadly refers to processes involving breaking up liquid mixtures into small droplets (atomization) and rapidly removing solvent from the mixture in a spray-drying apparatus (e.g., a nozzle) where there is a strong driving force for evaporation of solvent from the droplets. In a typical spray drying process, the feed liquid may be a solution, slurry, emulsion, gel or paste, provided it is pumpable and capable of being atomized.

In one embodiment, a pharmaceutical composition comprises a solid dispersion of the present disclosure further comprises one or more pharmaceutically acceptable excipients selected from binders, disintegrants, fillers, diluents, lubricants, glidants, surfactants, wetting agents, release rate modifiers, sweeteners, taste masking agents, colorants, flavors, and combinations thereof.

In one embodiment, a pharmaceutical composition of the present disclosure further comprises one or more filler and/or one or more binder and/or one or more disintegrant.

In one embodiment, the filler includes mannitol, microcrystalline cellulose, lactose, maltitol, dibasic calcium phosphate, sodium carboxymethycellulose, ethylcellulose, cellulose acetate, starch, glucose, fructose, sucrose, dicalcium phosphate, calcium sulfate, cellulose, kaolin, sodium chloride, sorbitol, trehalose, mantitol, lactitol, xylitol, isomalt, erythritol, and hydrogenated starch hydrolysates, or the like. In another embodiment, the filler includes mannitol, microcrystalline cellulose, lactose, maltitol, dibasic calcium phosphate, or the like. In another embodiment, the filler include D-mannitol, MCC 101, or a combination thereof.

In one embodiment, the binder includes hydroxypropyl methylcellulose (HPMC), polyethylene glycol (PEG), polyvinylpyrrolidone-vinylacetate copolymer (PVP-VA), povidone, polyvinyl pyrrolidone (PVP), hydroxypropyl cellulose (HPC), methyl cellulose, carboxy cellulose, polyvinyl alcohol, starch, sucrose, lactose, lactose monohydrate, maltitol, sorbitol, xylitol, poloxamer, gelatin, sugars, gums (such as xanthan gum, Arabic gum, or acacia gum), calcium hydrogen phosphate, dicalcium phosphate, glyceride, tragacanth, alginate, or the like. In another embodiment, the binder includes hydroxypropyl methylcellulose (HPMC), polyethylene glycol (PEG), polyvinylpyrrolidone-vinylacetate copolymer (PVP-VA), povidone, polyvinyl pyrrolidone (PVP), hydroxypropyl cellulose (HPC), methyl cellulose, or mixtures thereof. In some embodiment, the binder includes HPMC, HPC, PEG-4000, Povidone K30, PVP-VA64, or mixtures thereof.

In one embodiment, the disintegrant includes croscarmellose, crospovidone, copovidone, microcrystalline cellulose, hydroxypropylmethyl cellulose, sodium carboxymethyl starch, pregelatinized starch, sodium starch glycolate, starch, carboxymethyl cellulose (such as sodium carboxymethyl cellulose or calcium carboxymethyl cellulose), carmellose sodium, polacrilin potassium, alginate, or the like, but is not limited thereto. In another embodiment, the disintegrant includes croscarmellose, crospovidone, copovidone, microcrystalline cellulose, hydroxypropylmethyl cellulose, carboxymethyl starch, sodium starch glycolate, starch, carboxymethyl cellulose, alginate, or mixtures thereof, but is not limited thereto. In some embodiment, the disintegrant includes croscarmellose sodium, crospovidone, Starch 1500, or mixtures thereof, but is not limited thereto.

Suitable diluents include but not limited to lactose, mannitol, maltitol, glucose, hydroxypropyl cellulose, microcrystalline cellulose, starch, polyvinyl pyrrolidone, magnesium aluminate metasilicate, or the like.

Suitable lubricants include but not limited to magnesium stearate, calcium stearate, zinc stearate, stearic acid, stearyl alcohol, glyceryl monostearate, sodium stearyl fumarate, talc, glyceryl behenate, sodium benzoate, sodium lauryl sulfate, or the like.

Suitable surfactants include but not limited to sodium lauryl sulfate, monooleate, monolaurate, monopalmitate, monostearate or another ester of polyoxyethylene sorbitan, sodium dioctylsulfosuccinate (DOSS), lecithin, stearyl alcohol, cetostearylic alcohol, cholesterol, polyoxyethylene ricin oil, polyoxyethylene fatty acid glycerides, poloxamer, or any other commercially available co-processed surfactant like SEPITRAP® 80 or SEPITRAP® 4000, or the like.

Suitable sweeteners include but not limited to sucralose, aspartame, neotame, acesulfae-K, or the like.

Suitable glidants include but not limited to silicon dioxide, stearic acid, magnesium stearate, calcium hydroxide, talc, sodium stearyl fumarate, polyethylene glycol, polyethylene glycol, magnesium or sodium lauryl sulfate, colloidal silica, starch or the like.

The pharmaceutical composition comprises about 3%, 4%, 5%, 6%, 7%, 8%, 9%, 10%, 11%, 12%, %13%, 16%, 20%, 25%, 30%, 35%, or 40% by weight of the solid dispersion based on the total weight of the pharmaceutical composition. In one embodiment, the pharmaceutical composition comprises from about 3% w/w to about 40% w/w of the solid dispersion. In another embodiment, the pharmaceutical composition comprises from about 3% w/w to about 30% w/w of the solid dispersion. In other embodiment, the pharmaceutical composition comprises from about 3% w/w to about 20% w/w of the solid dispersion.

In one embodiment, the pharmaceutical composition comprising a solid dispersion can further comprise one or more filler. The pharmaceutical composition can comprise about 40%, 45%, 50%, 55%, 60%, 65%, 70%, 75%, 80%, 85%, 88%, 90%, 92%, or 95% by weight of the filler based on the total weight of the pharmaceutical composition. In one embodiment, the pharmaceutical composition comprises from about 40% w/w to about 95% w/w of the filler. In another embodiment, the pharmaceutical composition comprises from about 50% w/w to about 95% w/w of the filler. In other embodiment, the pharmaceutical composition comprises from about 50% w/w to about 92% w/w of the filler.

In one embodiment, the pharmaceutical composition comprising a solid dispersion and one or more filler can further comprise one or more binder and/or disintegrant. The pharmaceutical composition can comprise about 1%, 2%, 5%, 10%, 15%, 20%, 25%, 30%, or 35% by weight of the binder and/or disintegrant based on the total weight of the pharmaceutical composition. In one embodiment, the pharmaceutical composition comprises from about 1% w/w to about 35% w/w of the binder and/or disintegrant. In another embodiment, the pharmaceutical composition comprises from about 1% w/w to about 10% w/w of the binder and/or disintegrant. In other embodiment, the pharmaceutical composition comprises from about 10% w/w to about 30% w/w of the binder and/or disintergrant.

In one embodiment, the pharmaceutical composition comprising a solid dispersion can further comprise one or more filler, binder and disintegrant. The pharmaceutical composition can comprise about 50%, 55%, 60%, 65%, 70%, 75%, 80%, 85%, 88%, 90%, 92%, 95%, or 98% by weight of the filler, binder and disintegrant based on the total weight of the pharmaceutical composition. In one embodiment, the pharmaceutical composition comprises from about 50% w/w to about 98% w/w of the filler, binder and disintegrant. In another embodiment, the pharmaceutical composition comprises from about 70% w/w to about 95% w/w of the filler, binder and disintegrant. In other embodiment, the pharmaceutical composition comprises from about 80% w/w to about 92% w/w of the filler, binder and disintegrant.

In one embodiment, the pharmaceutical composition comprises from about 3% w/w to about 40% w/w of the solid dispersion, and from about 40% w/w to about 95% w/w of the filler. In another embodiment, the pharmaceutical composition comprised from about 3% w/w to about 30% w/w of the solid dispersion, and from about 50% w/w to about 95% w/w of the filler. In other embodiment, the pharmaceutical composition comprised from about 3% w/w to about 20% w/w of the solid dispersion, and from about 50% w/w to about 92% w/w of the filler.

In one embodiment, the pharmaceutical composition comprises from about 3% w/w to about 40% w/w of the solid dispersion, from about 40% w/w to about 95% w/w of the filler, and from about 1% w/w to about 35% w/w of the binder and/or disintergrant. In another embodiment, the pharmaceutical composition comprises from about 3% w/w to about 30% w/w of the solid dispersion, from about 50% w/w to about 95% w/w of the filler, and from about 1% w/w to about 30% w/w of the binder and/or disintergrant. In other embodiment, the pharmaceutical composition comprises from about 3% w/w to about 20% w/w of the solid dispersion, from about 50% w/w to about 92% w/w of the filler, and from about 2% w/w to about 30% w/w of the binder and/or disintergrant.

In one embodiment, the pharmaceutical composition is in a dosage form selected from the group consisting of a granule, an oral disintegrating tablet (ODT), a suspension, a powder, a solution, a granule or a powder for reconstitution as a suspension or a solution, a syrup, an elixir, a dispersible/effervescent tablet, a chewable tablet, a troche, an oral thin strip, a sachet, a pellet, a pill, a capsule, a sprinkle oral powder, or an inhaler.

In one embodiment, the pharmaceutical composition is a dosage form suitable for oral administration by pediatric patients.

The pharmaceutical composition provided herein can further be film-coated. The film-coating contains film-forming polymers and one or more coating additives. Suitable film-forming polymers include a cellulose derivative (e.g., methyl cellulose, hydroxymethyl cellulose, hydroxyethyl cellulose, hydroxypropyl cellulose, hydroxymethylethyl cellulose, hydroxypropylmethyl cellulose, sodium carboxymethyl cellulose, and ethyl cellulose), a vinyl polymer, an acrylic polymer, or a combination thereof.

The disclosure can be further understood by the following non-limiting examples.

### EXAMPLES

### Example 1

### Preparation of Compound A ([1-((11S)-7,8-difluoro(6H,11H-dibenzo[c,f]thiepin-11-yl))-4,6-dioxospiro[1,2,3,9-tetrahydropyridino[1,2-e]pyridazine-3,1′-cyclopropane]-5-yloxy]methyl methoxyformate)

Compound A was prepared by the synthetic route and protocols reported in Hsu et al., WO2019/144089. Shown below is the mass spectrum (MS) and nuclear magnetic resonance of Compound A: MS: m/z 541.0 (M++1); ¹H NMR (CDCl₃) δ7.31 (d, 1H), 7.06-7.00 (m, 4H), 6.85-6.84 (m, 1H), 6.73 (d, 1H), 6.03 (d, 1H), 5.96 (d, 1H), 5.80 (d, 1H), 5.49 (d, 1H), 5.15 (s, 1H), 4.13 (d, 1H), 4.05 (d, 1H), 3.87 (s, 3H), 2.91 (d, 1H), 1.95-1.90 (m, 1H), 1.49-1.48 (m, 1H), 0.88-0.76 (m, 2H). In addition, 1′-((11S)-7,8-difluoro-6,11-dihydrodibenzo[b,e]thiepin-11-yl)-1′,2′-dihydro-5′-hydroxy-spiro[cyclopropane-1,3′-(3H)pyrido[1,2-b]pyridazine-4′,6′-dione, the metabolite of Compound A, was also prepared by the similar synthetic route and protocols. Mass spectrum (MS) and nuclear magnetic resonance of the metabolite of Compound A are shown as follows: MS: m/z 453.1 (M+H)+; ¹H NMR (CDCl₃) δ7.27 (d, 1H), 7.08-7.00 (m, 4H), 6.80-6.78 (m, 1H), 6.63 (d, 1H), 5.83 (d, 1H), 5.50 (dd, 1H), 5.23 (s, 1H), 4.15 (d, 1H), 4.06 (d, 1H), 2.91 (d, 1H), 2.43 (br, 1H), 1.92-1.84 (m, 1H), 1.78-1.67 (m, 1H), 0.96-0.92 (m, 1H), 0.86-0.81 (m, 1H)

### Example 2

### Preparation of Amorphous Solid Dispersions

The amorphous solid dispersions of this disclosure were prepared by spray drying well known in the art. For example, see Singh et al., Advanced Drug Delivery Reviews, 2016, 100, 27-50. Different pharmaceutically acceptable polymer, such as PVP-VA64, Soluplus, HPMCAS-MG, Eudragit EPO, HPC-SSL, HPMCP HP-55, Kollidon SR, and PEG-3350, were used for making the amorphous solid dispersions of this disclosure. Spray dryer 4M8-Trix was used to prepare the amorphous solid dispersions. The spray drying concentration of Compound A was set at 25 mg/ml. Compound A was mixed with different pharmaceutical acceptable polymers at various ratios and dissolved in the solvents, such as acetone, in glass bottles to provide a feeder solution. The obtained feeder solution was passed through a nozzle as a fine spray into a chamber where the solvent was evaporated quickly to generate particles containing Compound A and a corresponding polymer. The resulting spray dried powder was dried further to remove residual solvents in a static dryer.

### Example 3

### Dissolution Assessments

An amount of Compound A (about 6 mg) and amorphous solid dispersion (equivalent to around 6 mg Compound A) were weighed into an 8 ml bottle, and 6 ml FaSSIF (Fasted state simulated intestinal fluid) was added (target concentration 1.0 mg/ml). Then the suspensions were stirred at thermomixer at 37° C. under 600 rpm 200 μL of suspensions were withdrawn at setting time (such as 5 min) and then centrifuged at 14000 rpm for 4 min. After that, 100 μL of supernatant was diluted with CAN five times to prevent precipitation, and was analyzed by HPLC. Compound A have low aqueous solubility.

FaSSIF was prepared by following steps: 1) Place 0.1024 g sodium hydroxide, 0.7518 g sodium dihydrogen phosphate anhydrous and 1.5470 g sodium chloride in a 250 mL volumetric flask, add about 225 mL water, and adjust the pH to 6.5 with 1N sodium hydroxide or 1N hydrochloric acid. Make up to volume (250 mL) with pure water. 2) Place and dissolve 0.4480 g SIF Powder Original with 100 mL of buffer (from step 1) in a 200 mL volumetric flask, add water to volume and mix well.

TABLE 1 listed the solubility of Compound A and amorphous solid dispersions were collected manually at 5 and 15 min in FaSSIF.

TABLE 1
Solubility of Compound A and amorphous solid dispersion thereof

| Sample | | | | Ratio | Solubility (μg/mL) | |
|---|---|---|---|---|---|---|
| No. | Formulation | Drug | Polymer | (Drug:Polymer) | 5 min | 15 min |
| 1 | — | Compound A | — | — | 4.95 | 4.89 |
| 2 | ASD | Compound A | HPMCP HP-55 | 1:1 | 64.60 | 12.27 |
| 3 | ASD | Compound A | Kollidon SR | 1:1 | 16.65 | 12.46 |
| 4 | ASD | Compound A | PEG3350 | 1:1 | 6.63 | 6.51 |
| 5 | ASD | Compound A | PVP-VA64 | 1:1 | 115.30 | 10.82 |
| 6 | ASD | Compound A | Soluplus | 1:1 | 131.80 | 130.00 |
| 7 | ASD | Compound A | Soluplus | 1:2 | 138.30 | 136.00 |
| 8 | ASD | Compound A | Eudragit EPO | 1:1 | 65.73 | 73.08 |
| 9 | ASD | Compound A | HPMCAS-MG | 1:1 | 130.90 | 133.40 |
| 10 | ASD | Compound A | HPMCAS-HG | 1:1 | 100.40 | 102.90 |
| 11 | ASD | Compound A | HPC-SSL | 1:1 | 112.80 | 119.10 |
| 12 | ASD | Compound A | HPC-SSL | 1:3 | 140.00 | — |
| 13 | ASD | Compound A | HPC-SSL | 1:5 | 126.30 | — |
| 14 | ASD | Compound A | HPC-SSL | 1:1.5 | 125.80 | 118.90 |

### Example 4

### Stability Study of Compound A and Amorphous Solid Dispersions

The amorphous solid dispersions were stored at 4° C. (closed) or 40° C./75% RH (closed and open state). After 10 days or 4 weeks, samples were observed by camera, characterized by XRPD and analyzed by HPLC for purity as well as kinetic solubility. All the samples for closed storage conditions were put into clear glass vials with gaskets and screwed caps. All the samples for open storage conditions were put into clear glass vials with cap, while the month of vials was covered by aluminium foil with pinholes to avoid cross-contamination.

TABLE 2 showed total impurity level (i.e., TRS %, total relative substances) of Compound A and ASDs after stability tests. The sample number in TABLE 2 corresponds to the sample number in TABLE 1.

TABLE 2

| | | | 4° C. | 40° C./75% RH | |
|---|---|---|---|---|---|
| | | | Closed for | Closed for | Open for |
| Sample | | Initial | 4 wks | 4 wks | 10 d ^{#}/4 wks |
| No. | polymer | TRS % | TRS % | TRS % | TRS % |
| 1 | — | 0.79 | 0.76 | 0.86 | 0.82 |
| 2 | HPMCP | 0.90 | — | 4.72 | 5.58 |
| | HP-55 | | | | |
| 4 | PEG-3350 | 0.82 | — | 0.86 | 0.82 |
| 5 | PVP-VA64 | 0.80 | 0.78 | — | 1.50 |
| 6 | Soluplus | 0.78 | 0.77 | 0.92 | 1.22 |
| 8 | Eudragit | 0.91 | 1.46 | — | 45.31 ^{#} |
| | EPO | | | | |
| 9 | HPMCAS- | 0.78 | 0.86 | — | 2.08 ^{#} |
| | MG | | | | |
| 10 | HPMCAS- | 0.78 | | | 1.52 ^{#} |
| | HG | | | | |
| 11 | HPC-SSL | 0.77 | 0.78 | — | 1.35 |

### Example 5

### Formulation and Preparation

The Formulations comprising solid dispersions of Compound A were as shown in Table 3 and 4. The amorphous solid dispersions (ASD) comprising Compound A were as disclosed in Example 2. Formulations of granule were prepared by a Roller Compactor. ASD and equal amount mannitol were mixed manually for 2 minutes, and then mannitol was added to the bag of ASD shaking for 2 minutes to clear residual ASD. Then the other excipients were added by the method of increment by equal quantity. Finally, the mixture was mixed by a Shaker Mixer turbula for 10 minutes at 46 rpm/min.

TABLE 3

| | | Granule Formulation | | | | | | | | | |
|---|---|---|---|---|---|---|---|---|---|---|---|
| | | G1 | G2 | G3 | G4 | G5 | G6 | G7 | G8 | G9 | G10 |
| | | weight | | | | | | | | | |
| Component | | mg | mg | mg | mg | mg | mg | mg | mg | mg | mg |
| ASD | Compound A | 10 | 10 | 10 | 10 | 10 | 10 | 10 | 10 | 10 | 20 |

| | | | | | | | | | | |
|---|---|---|---|---|---|---|---|---|---|---|
| HPC-SSL | 30 | 30 | 30 | 30 | 30 | 30 | 30 | 30 | 30 | 60 |
| D-mannitol | 220 | 438 | 428 | 438 | 403 | 220 | 435 | 435 | 435 | 398 |
| MCC 101 | 233 | | | | 50 | 235 | | | | |
| HPMC | | | | | | | 20 | | | |
| PEG-4000 | | | | | | | | 20 | | |
| Povidone K30 | | 15 | 15 | | | | | | 15 | 15 |
| PVP-VA64 | | | | 15 | | | | | | |
| Croscarmellose sodium | | | 10 | | | | | | | |
| Sucralose | 2 | 2 | 2 | 2 | 2 | | | | 2 | 2 |
| SiO₂ | | | | | | | 5 | 3.5 | | |
| Magnesium stearate | 5 | 5 | 5 | 5 | 5 | 5 | | 1.5 | 5 | 5 |
| Total weight | 500 | 500 | 500 | 500 | 500 | 500 | 500 | 500 | 500 | 500 |

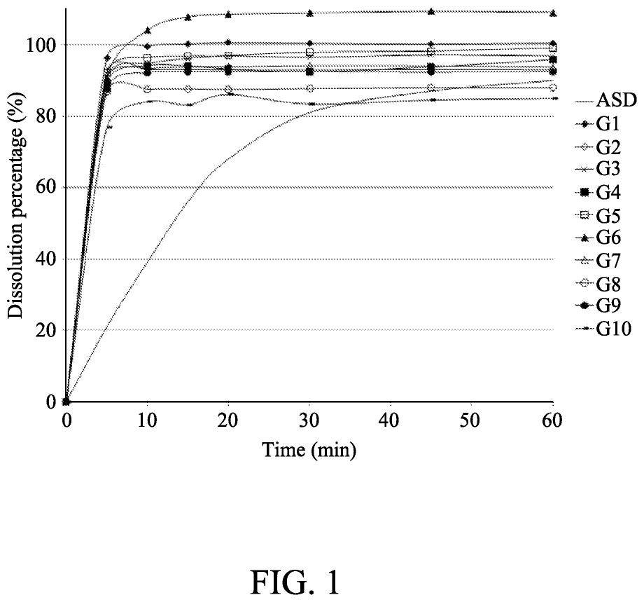

Dissolution profile of the granule formulations and ASD powder itself were shown in FIG. 1. The tested granule formulations achieved about 80% dissolution within 15 minutes.

TABLE 4

| | | ODT formulation | | | | | | | | |
|---|---|---|---|---|---|---|---|---|---|---|
| | | D1 | D2 | D3 | D4 | D5 | D6 | D7 | D8 | D9 |
| | | weight | | | | | | | | |
| Component | | mg | mg | mg | mg | mg | mg | mg | mg | mg |
| ASD | Compound A | 10 | 10 | 10 | 10 | 10 | 10 | 10 | 10 | 10 |

| | | | | | | | | | |
|---|---|---|---|---|---|---|---|---|---|
| HPC-SSL | 30 | 30 | 30 | 30 | 30 | 30 | 30 | 30 | 30 |
| D-mannitol | 60.3 | 60.3 | 42.3 | 300 | 300 | 300 | 300 | 300 | 200 |
| MCC 101 | 12 | 12 | 12 | 40 | 40 | | 40 | 40 | |
| Croscarmellose sodium | 6 | 24 | 24 | | 14 | 14 | 14 | 14 | |
| Crospovidone | | | 13.4 | 43.5 | 43.5 | 45 | 45 | 45 | 18 |
| Starch 1500 | | | | | | 40 | | | 80 |
| Sucralose | 0.5 | 0.6 | 0.5 | 0.5 | 0.5 | 0.5 | 0.5 | 0.5 | 0.5 |
| Magnesium stearate | 1.2 | 1.2 | 1.2 | 4.5 | 4.5 | 4.5 | 4.5 | 4.5 | 3.5 |
| Total weight | 120 | 138.1 | 133.4 | 428.5 | 442.5 | 444 | 362 | 444 | 342 |

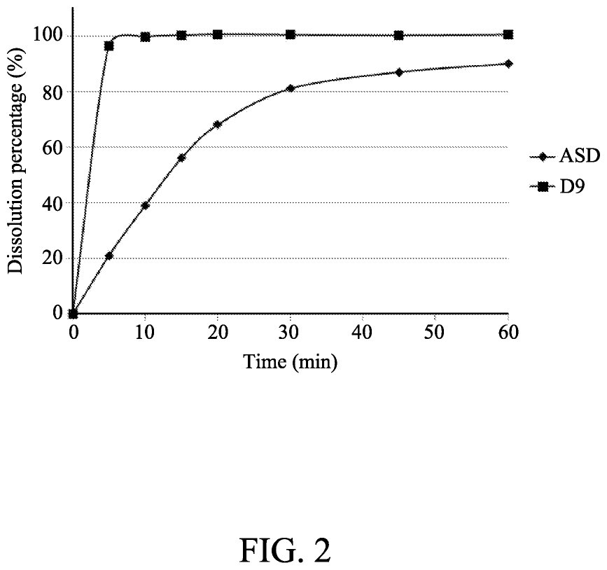

Dissolution profile of the D9 formulation and ASD powder itself were shown in FIG. 2. The tested formulation achieved about 80% dissolution within 15 minutes.

### OTHER EMBODIMENTS

All of the features disclosed in this specification may be combined in any combination. Each feature disclosed in this specification may be replaced by an alternative feature serving the same, equivalent or similar purpose. Thus, unless expressly stated otherwise, each feature disclosed is only an example of a series of equivalent or similar features.

From the above description, one skilled in the art can easily ascertain the essential characteristics of the present disclosure, and without departing from the scope thereof, can make various changes and modifications of the disclosure to adapt it to various usage and conditions. Thus, other embodiments are also within the scope of the following claims.

What is claimed is:

1. A pharmaceutical composition comprising a solid dispersion, wherein the solid dispersion comprises:
i) a compound of Formula (II) or a pharmaceutically acceptable salt thereof: 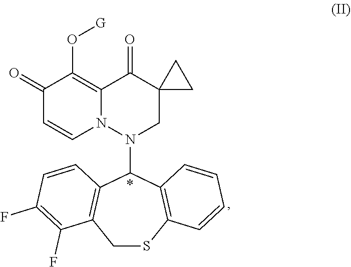
wherein G is hydrogen or —C(R₂R₂′)—O—CO—O—R₃, in which each of R₂, and R₂′, independently, is hydrogen or C₁₋₄ alkyl; R₃ is C₁₋₄ alkyl; the star (*) indicates a chiral center; and
ii) a pharmaceutically acceptable polymer,
wherein a weight ratio of the compound of Formula (II) or a pharmaceutically acceptable salt thereof to the pharmaceutically acceptable polymer is from about 1:1 to about 1:5, and
wherein the compound of Formula (II) or a pharmaceutically acceptable salt thereof is in a therapeutically effective amount from about 5 mg to about 200 mg.

2. The pharmaceutical composition according to claim 1, wherein the compound of Formula (II) is [1-((11S)-7,8-difluoro(6H,11H-dibenzo[c,f]thiepin-11-yl))-4,6-dioxospiro[1,2,3,9-tetrahydropyridino[1,2-e]pyridazine-3,1′-cyclo propane]-5-yloxy]methyl methoxyformate, or 1′-((11S)-7,8-difluoro-6,11-dihydrodibenzo[b,e]thiepin-11-yl)-1′,2′-dihydro-5′-hydroxy-spiro[cyclopropane-1,3′-(3H)pyrido[1,2-b]pyridazine-4′,6′-dione;
wherein the pharmaceutically acceptable polymer is polyvinyl caprolactam-polyvinyl acetate-polyethylene glycol graft copolymer (Soluplus), hydroxypropyl cellulose (HPC), hydroxypropyl methyl cellulose acetate succinate (HPMCAS), or mixtures thereof.

3. The pharmaceutical composition according to claim 2, wherein the pharmaceutically acceptable polymer is Soluplus, HPC-SSL, HPMCAS-MG, HPMCAS-HG, or mixtures thereof.

4. The pharmaceutical composition according to claim 1, wherein the weight ratio of the compound of Formula (II) or a pharmaceutically acceptable salt thereof to the pharmaceutically acceptable polymer is from about 1:1 to about 1:3.

5. The pharmaceutical composition according to claim 1, wherein the compound of Formula (II) or a pharmaceutically acceptable salt thereof is in a therapeutically effective amount from about 5 mg to about 100 mg.

6. The pharmaceutical composition according to claim 1, wherein the solid dispersion is present at a concentration from about 3% w/w to about 40% w/w.

7. The pharmaceutical composition according to claim 1, wherein the solid dispersion has a D₅₀ particle size in a range of about 4 μm to about 15 μm, or a D₉₀ particle size in a range of about 15 μm to about 50 μm.

8. The pharmaceutical composition according to claim 1, wherein the pharmaceutical composition further comprises one or more filler.

9. The pharmaceutical composition according to claim 8, wherein the pharmaceutical composition further comprises one or more binder and/or disintegrant.

10. The pharmaceutical composition according to claim 9, wherein the filler is selected from the group consisting of mannitol, microcrystalline cellulose, lactose, maltitol, dibasic calcium phosphate, sodium carboxymethycellulose, ethylcellulose, cellulose acetate, starch, glucose, fructose, sucrose, dicalcium phosphate, calcium sulfate, cellulose, kaolin, sodium chloride, sorbitol, trehalose, mantitol, lactitol, xylitol, isomalt, erythritol, and hydrogenated starch hydrolysates, and a combination thereof; wherein the binder is selected from the group consisting of hydroxypropyl methylcellulose (HPMC), polyethylene glycol (PEG), polyvinylpyrrolidone-vinylacetate copolymer (PVP-VA), povidone, polyvinyl pyrrolidone (PVP), hydroxypropyl cellulose (HPC), methyl cellulose, and a combination thereof; and wherein the disintegrant is selected from the group consisting of croscarmellose, crospovidone, copovidone, microcrystalline cellulose, hydroxypropylmethyl cellulose, carboxymethyl starch, sodium starch glycolate, starch, carboxymethyl cellulose, alginate, and a combination thereof.

11. The pharmaceutical composition according to claim 10, wherein the filler is selected from the group consisting of D-mannitol, MCC 101, and a combination thereof; wherein the binder is selected from the group consisting of HPMC, HPC, PEG-4000, Povidone K30, PVP-VA64, and a combination thereof; and wherein the disintegrant is selected from the group consisting of croscarmellose sodium, crospovidone, Starch 1500, and a combination thereof.

12. The pharmaceutical composition according to claim 8, wherein the solid dispersion is present at a concentration from about 3% w/w to about 40% w/w, and wherein the filler is present at a concentration from about 40% w/w to about 95% w/w.

13. The pharmaceutical composition according to claim 9, wherein the solid dispersion is present at a concentration from about 3% w/w to about 40% w/w, the filler is present at a concentration from about 40% w/w to about 95% w/w, and the binder and/or disintegrant is present at a concentration from about 1% w/w to about 35% w/w.

14. The pharmaceutical composition according to claim 9, wherein the pharmaceutical composition further comprises one or more pharmaceutically acceptable excipients selected from the group consisting of diluents, lubricants, glidants, surfactants, wetting agents, release rate modifiers, sweeteners, taste masking agents, colorants, and flavors.

15. The pharmaceutical composition according to claim 1, wherein the pharmaceutical composition is in a dosage form selected from the group consisting of a granule, an oral disintegrating tablet (ODT), a suspension, a powder, a solution, a granule or a powder for reconstitution as a suspension or a solution, a syrup, an elixir, a dispersible/effervescent tablet, a chewable tablet, a troche, an oral thin strip, a sachet, a pellet, a pill, a capsule, a sprinkle oral powder, and an inhaler form.
